(19) United States
(12) Patent Application Publication
(10) Pub. No.: US 2009/0264443 A1
(43) Pub. Date: Oct. 22, 2009

(54) TREATMENT OF ORGANOPHOSPHATE EXPOSURE WITH TETRAHYDROINDOLONE ARYLPIPERAZINE COMPOUNDS

(76) Inventors: David HELTON, Foothill Ranch, CA (US); David FICK, Coto de Caza, CA (US)

Correspondence Address: SHELDON MAK ROSE & ANDERSON PC, 100 Corson Street, Third Floor, PASADENA, CA, 91103-3842, US

(21) Appl. No.: 12/105,608

(22) Filed: Apr. 18, 2008

(51) Int. Cl. A61K 31/497; A61P 43/00

(52) U.S. Cl. 514/254.09

(57) ABSTRACT

A method of treating exposure to organophosphate agents through the use of compounds comprising tetrahydroindolone and arylpiperazine moieties.

### BACKGROUND

Organophosphate compounds, in particular organic esters of substituted phosphoric acids, have been developed for use as chemical weapons. These compounds inhibit cholinesterases and disrupt the peripheral nervous system by preventing these enzymes from breaking down acetylcholine. Some organophosphate compounds are sufficiently potent that even brief exposure may be fatal.

Organophosphate anticholinesterase agents include tabun (Ethyl N,N-dimethylphosphoramidocyanidate, also referred to as GA), sarin (O-Isopropyl methylphosphonofluoridate, also referred to as GB), soman (O-Pinacolyl methylphosphonofluoridate, also referred to as GD), and VX (O-ethyl-S-[2(diisopropylamino)ethyl]methylphosphonothiolate). Tabun, sarin, and soman in particular are highly volatile and easily disseminated in vapor form. They are also readily absorbed through the lungs, eyes, skin, and intestinal tract.

Individuals who survive exposure to organophosphate agents may experience morbidity as a result of such exposure. Some survivors of sarin exposure, for example, have exhibited conditions including post traumatic stress syndrome, memory deficits and altered evoked potentials (Murata K, Araki S, Yokoyama K, Okumura T, Ishimatsu S, Takasu N and White R F, Asymptomatic sequelae to acute sarin poisoning in the central and autonomic nervous system 6 months after the Tokyo subway attack, J Neurol 244: 601-606, 1997). Munitions workers exposed to organophosphate agents in the U.S. demonstrated EEG changes, while a similar population in Russia showed long lasting memory loss, sleep disorders and neurological impairments (Romano J A, McDonough J H Jr, Sheridan R E and Sidell F R. “Health Effects of Low-Level Exposure to Nerve Agents,” Chemical Warfare Agents: Toxicity at Low Levels, edited by Somani S M and Romano J A, CRC Press, 2001, pp. 1-24; Duffy F H, Burchfiel J L, Bartels P H, Gaon M and Sim V M, “Long-Term Effects of An Organophosphate Upon the Human Encephalogram,” Toxicology and Applied Pharmacology, 1979, 47: 161-176).

No effective therapies currently exist for treating the long-term effects of exposure to organophosphate agents in individuals who survive such exposure. In addition, the current standard of care for treating acute organophosphate exposure, namely the injection of atropine, carries a risk of adverse reactions. In view of the threat posed by organophosphate agents, improved therapies for treating individuals exposed to such agents and for preventing the harm that these agents can cause are needed.

### SUMMARY

The present compounds act as neuroprotective agents with respect to the toxicity associated with exposure to organophosphorus nerve agents such as soman, tabun, VX and sarin. These compounds can be used to treat individuals who have been exposed to such agents, and can also be administered to individuals at risk for exposure to nerve agents prior to such exposure.

The present method of treating the effects of exposure to an organophosphate compound comprises administering to a subject in need thereof a therapeutically effective amount of a pharmaceutical composition that includes a compound which preferably has the following formula (Formula I):

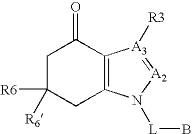

where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl; and
(d) R_{6′} is hydrogen unless R₆ is alkyl, in which case R_{6′} is hydrogen or the same alkyl as R₆.
(e) L is a linker; and
(f) B is a moiety having a formula selected from the group consisting of:
   (i) Formula II:

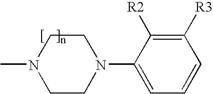

where:
   (1) R2 is hydrogen, alkyl, hydroxy, halo, alkoxy, cyano, methylthio;
   (2) R3 is hydrogen, alkyl, hydroxy, halo, alkoxy, trifluoromethyl, nitro, amino, aminocarbonyl, aminosulfonyl;
   (3) R2 and R3 can be taken together to form a 5 or 6 member aromatic or non-aromatic ring, which can contain from 0 to 3 heteroatoms selected from the group of N, O, or S of which the N may be further substituted if in a non-aromatic ring; and
   (4) n equals 1 or 2;
(ii) Formula III:

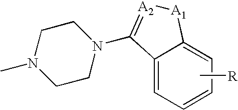

where:
   (1) A1 is N, O, or S, and when it is N, it can be further substituted with Z, which in alkyl, aralkyl, heteroaralky, or heteroalkyl.
   (2) A2 is C or N;
   (3) n is 1 or 2; and
   (4) R is selected from the group consisting of hydrogen, alkyl, NH₂, NHQ₁, NQ₁Q₂, OH, OQ₁, SQ₁, halo, nitro, cyano, and trifluoromethyl, and wherein Q₁ and Q₂ are selected from the group consisting of alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl; and
(iii) Formula IV:

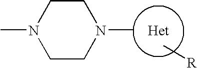

where the 6-member heterocyclic ring of Formula IV is selected from the group consisting of a 2-pyridyl moiety, a 4-pyridyl moiety, a 2-pyrimidyl moiety, a 4-pyrimidyl moiety, a 2-pyrazinyl moiety, and a 2-triazinyl moiety;

or a salt or ester thereof. Preferably, A₂ and A₃ are C; R₆ is hydrogen, alkyl, aralkyl, heteroaralkyl, aryl or heteroaryl; and R₆ and R₃ are hydrogen. More preferably, the compound of Formula I is a compound from Table 1 below.

The linker in the present compounds can be, for example, a straight chain alkyl group having the formula —(CH₂)ₘ—, wherein m is an integer from 1 to 6, or can be an alkyl substituted hydrocarbyl moiety having the following formula:

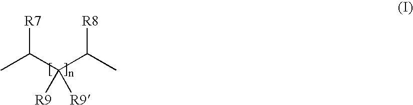

where:
(i)ₙ is 0, 1 or 2;
(ii) R7 and R8 are hydrogen, methyl or ethyl;
(iii) R9 and R9′ are both hydrogen, methyl or ethyl;
(iv) if n is 1 and R7 or R8 is methyl or ethyl, then R9 and R9′ are hydrogen;
(v) if n is 1 and R7 and R8 are hydrogen, then R9 and R9′ are methyl or ethyl; and
(vi) if n is 2, then R9 and R9′ are hydrogen and one or both of R7 and R8 are methyl or ethyl.

The B moiety of the present compounds is preferably either a m-trifluoromethylphenylpiperazinyl moiety, a m-chlorophenylpiperazinyl moiety, a o-methoxyphenylpiperazinyl moiety, a 1-naphthylpiperazinyl moiety, a 2-pyrimidylpiperazinyl moiety, a 3-indazolylpiperazinyl moiety a 2,3-dichlorophenylpiperazinyl moiety, or a 2,3-dimethylphenylpiperazinyl moiety. The R group of the B moiety is also preferably a halo group, an alkyl group, a cyano group, a trifluoromethyl group, an alkoxy group, an amino group, an alkylamino group, or a dialkyamino group. In preferred embodiments, the B moiety can be:

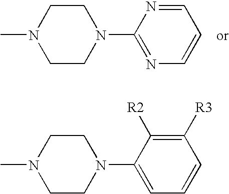

and R₂ and R₃ are the same or independently hydrogen, alkyl, hydroxy, halo, alkoxy, trifluoromethyl, nitro, amino, aminocarbonyl, or aminosulfonyl.

The composition used in the present methods also preferably comprises a pharmaceutically acceptable excipient in combination with the compound of Formula I, and is formulated for administration intravenously, orally, topically, intraperitoneally, intravesically, transdermally, nasally, rectally, vaginally, intramuscularly, intradermally, subcutaneously and/or intrathecally. A therapeutically effective amount of the compound of Formula I is preferably in the range of 0.0001 mg/kg to 60 mg/kg of a subject's weight.

In the present methods, the present compounds can be administered either before or after exposure of a subject to an organophosphate compound. The present compounds can thus act as prophylactic treatments or as treatments following exposure to such a compound.

### DESCRIPTION

### Definitions

As used herein, the following terms and variations thereof have the meanings given below, unless a different meaning is clearly intended by the context in which such term is used.

“Alkyl” refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “heteroalkyl” refers to carbon-containing straight-chained, branch-chained and cyclic groups, all of which can be optionally substituted, containing at least one O, N or S heteroatom. The term “alkoxy” refers to the ether —O-alkyl, where alkyl is defined as above.

“Alkenyl” refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “heteroalkenyl” refers to unsaturated groups which contain at least one carbon-carbon double bond and includes straight-chained, branch-chained and cyclic groups, all of which can be optionally substituted, containing at least one O, N or S heteroatom.

“Aryl” refers to aromatic groups that have at least one ring having a conjugated, pi-electron system and includes carbocyclic aryl and biaryl, both of which can be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above.

“Central nervous system” refers to the part of the nervous system that includes the brain and spinal cord. The central nervous system does not include the peripheral nerves which carry signals between the central nervous system and the muscles and organs of the body.

“Derivative” refers to a compound that is modified or partially substituted with another component.

“Hydrocarbyl” refers to a hydrocarbon chain, which can be optionally substituted or provided with other substitutions known to the art.

“Optionally substituted” refers to one or more substituents which can be, without limitation, alkyl, aryl, amino, hydroxy, alkoxy, aryloxy, alkylamino, arylamino, alkylthio, arylthio, or oxo, cyano, acetoxy, or halo moieties.

“Organophosphate compounds” refer to esters of phosphoric acid which act on the enzyme acetylcholinesterase and have neurotoxicity. Such compounds include nerve agents such as tabun (Ethyl N,N-dimethylphosphoramidocyanidate, also referred to as GA), sarin (O-Isopropyl methylphosphonofluoridate, also referred to as GB), soman (O-Pinacolyl methylphosphonofluoridate, also referred to as GD), and VX (O-ethyl-S-[2(diisopropylamino)ethyl]methylphosphonothiolate), as well as some compounds used as insecticides, such as phosphoric acid diethyl 4-nitrophenyl ester (paraoxon), diethyl-p-nitrophenyl monothiophosphate (parathion) and phosphorothioic acid O-(3-chloro-4-methyl-2-oxo-2H-1-benzopyran-7-yl) O,O-diethyl ester (coumaphos).

A “subject” refers a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

“Sulfonyl” refers to the group —S(O₂)—. The term “halo” refers to fluoro-, chloro-, bromo-, or iodo-substitutions. The term “alkanoyl” refers to the group —C(O)R, where R is alkyl. The term “aroyl” refers to the group —C(O)R, where R is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group.

“Treat” and “treatment,” with respect to the exposure of a subject to an organophosphate compound, refer to a medical intervention which attenuates, prevents, and/or counteracts the effects of such exposure. The foregoing terms can refer to the prophylactic administration of the present compounds and compositions to subjects at risk of exposure to an organophosphate compound prior to an anticipated exposure, and/or can refer to the administration of the present compounds and compositions following such exposure.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Compounds

The present compounds have the general schematic structure {A}-L-{B}, where the A moiety is a bicyclic ring structure such as tetrahydroindolone or a tetrahydroindolone derivative, L is a hydrocarbyl chain linker, and the B moiety is an arylpiperazine or arylpiperazine derivative, as described below.

### Tetrahydroindolone Moiety

In one embodiment, the A moiety of the present compounds is an 8-10 atom bicyclic moiety in which the five-aromatic membered ring has 1 to 2 nitrogen atoms, the bicyclic moiety having the structure of formula (I):

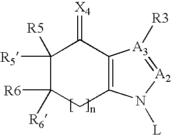

where:
(a) formula I is bonded to a hydrocarbyl linker L;
(b) A₂ and A₃ are C or N;
(c) R₃ is hydrogen, alkyl, aralky, heteroaralkyl, heteroalkyl, alkenyl, aralkenyl, heteroaralkenyl, heteroalkenyl, aryl, or heteroaryl;
(d) X₄ is O, S or N—OH;
(e) R₅ is hydrogen, alkyl, aralkyl, heteroaralkyl, alkanoyl, aroyl, heteroaroyl, aralkanoyl, heteroaralkanoyl, NH₂, NH Q₁, NQ₁Q₂, OH, OQi, or SQi, where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(f) R₅ is hydrogen unless R₅ is alkyl, in which case R₅ is hydrogen or the same alkyl as R₅;
(g) R₅ and R_{5′} can be taken together as a double bond to C₅ and can be O, S, NQ₃, or C which can be substituted with one or two groups R₅, where Q3 is alkyl, aralkyl, heteroaralkyl, aryl, heteroaryl, hydroxy, alkoxy, aryloxy, or heteroaryloxy in which the alkyl portions can be cyclic and can contain from 1 to 3 heteroatoms which can be N, O, or S;
(h) R₆ is hydrogen, alkyl, aryl, heteroaryl;
(i) R_{6′} is hydrogen unless R₆ is alkyl, in which case R_{6′} is hydrogen or the same alkyl as R₆; and
(j)n is 0 to 2.

As shown in Formula (I), the moiety A has a five, six, or seven-membered saturated ring fused to a five-membered aromatic ring. The five-membered aromatic ring can have one or two nitrogen atoms as indicated, but the five-membered aromatic ring always has a nitrogen atom at the 1-position. Typically, the five-membered aromatic ring has one nitrogen atom as in tetrahydroindolone. This nitrogen atom at the 1-position is covalently bonded to the linker L. Typically A is a tetrahydroindolone moiety in which A₂ is carbon and n is 1. The tetrahydroindolone moiety can be variously substituted.

In another embodiment, A is a tetrahydroindolone moiety. One example of a tetrahydroindolone moiety for the moiety A is a tetrahydroindolone moiety of Formula (II) below:

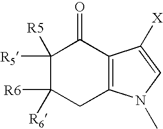

where:
(1) X is H or CH₂N(CH₃)₂;
(2) R₅ is hydrogen, alkyl, aralkyl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q1 and Q2 are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and where W₁ is alkyl, aralkyl, heteroaralkyl, aryl, heteroaryl, alkanoyl, aroyl, aralkanoyl, heteroaralkanoyl, or heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S;
(3) R_{5′} is hydrogen;
(4) R₆ is hydrogen, alkyl, aryl, heteroaryl; and
(5) R_{6′} is hydrogen.
The tetrahydroindolone of Formula II is bonded to a linker L as in Formula I above. In one embodiment, R₅, R_{5′}, R₆, and R_{6′}, are all hydrogen. In this embodiment, the moiety A is thus an unsubstituted tetrahydroindolone moiety.

In another embodiment in which the A moiety is a tetrahydroindolone moiety, the A moiety can be a tetrahydroindolone of Formula (III):

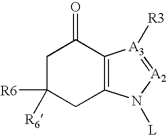

where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl; and
(d) R_{6′} is hydrogen unless R₆ is alkyl, in which case R_{6′} is hydrogen or the same alkyl as R₆.

The tetrahydroindolone of Formula III is bonded to a linker L as in Formula I above.

### Arylpiperazine Moiety

The B moiety of the present compounds is an arylpiperazine or derivative having the structure of formula (IV):

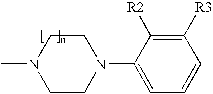

where:
(a) R2 is hydrogen, alkyl, hydroxy, halo, alkoxy, cyano, methylthio;
(b) R3 is hydrogen, alkyl, hydroxy, methoxy, halo, alkoxy, trifluoromethyl, nitro, amino, aminocarbonyl, aminosulfonyl;
(c) R2 and R3 can be taken together to form a 5 or 6 member aromatic or non-aromatic ring, which can contain from 0 to 3 heteroatoms selected from the group of N, O, or S; and
(d) n equals 1 or 2.

Preferably, the aryl piperazine moiety comprises one or more of the following substitutions:
(i) R₄ is alkyl, halo, alkoxy, or perfluoroalkyl;
(ii) R₃ and R when taken together are either a methylenedioxy or ethylenedioxy group.

In one embodiment, B is a m-trifluoromethylphenylpiperazinyl moiety:

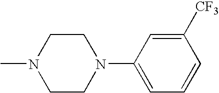

In another embodiment, B is a m-chlorophenylpiperazinyl moiety:

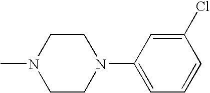

In yet another embodiment, B is an o-methoxyphenylpiperazinyl moiety:

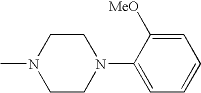

In another embodiment, B is a piperazine ring or derivative linked to a 6-member heterocyclic ring containing 1 to 3 N, having the structural formula (V):

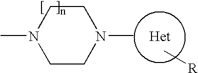

where n=1 or 2 and the 6-member heterocyclic ring (Het) can be 2-pyridyl, 4-pyridyl, 2-pyrimidyl, 4-pyrimidyl, 2-pyrazinyl, 2-triazinyl, 2,3-dichlorophenylpiperazinyl, or 2,3-dimethylphenylpiperazinyl. The heterocyclic ring can also be substituted where R can be halo, alkyl, cyano, trifluoromethyl, alkoxy, amino, alkylamino, or dialkyamino.

In one embodiment of the foregoing piperazine derivative, B is a 2-pyrimidylpiperazinyl moiety:

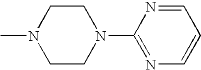

In another embodiment, B is a 1-pyrimidin-2-yl-[1,4]diazepane moiety:

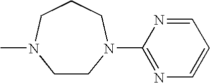

In another embodiment, B is piperazine ring or derivative linked to a bicyclic moiety having the structure (VI) below:

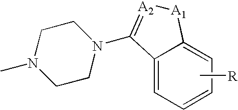

where:
(a) A1 is N, O, or S, and when it is N, it can be further substituted with Z, which in alkyl, aralkyl, heteroaralky, or heteroalkyl.
(b) A2 is C or N;
(c) n is 1 or 2; and
(d) R is hydrogen, alkyl, NH2, NHQ1, NQ1 Q2, OH, OQ1, SQ1, halo, nitro, cyano, or trifluoromethyl where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which may contain 1 other heteroatom which can be N, O, or S, of which the N may be further substituted with Y2, where Y2 is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

In another embodiment, B is a piperazine ring or derivative linked to a bicyclic moiety having the structural formula (VII):

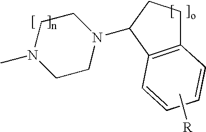

where:
(a) o is 1 to 3;
(b) n is 1 or 2; and
(c) R is hydrogen, alkyl, NH2, NHQ1, NQ1 Q2, OH, OQ1, SQ1, nitro, cyano, trifluoromethyl, or halo where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which can contain 1 other heteroatom which can be N, O, or S, of which the N can be further substituted with Y2, where Y2 is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

In another embodiment, B is an arylpiperazine or derivative having the structure of formula (VIII):

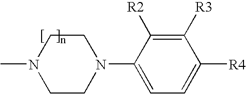

where:
(a) R2 is hydrogen, alkyl, hydroxy, halo, alkoxy, cyano, methylthio;
(b) R3 is hydrogen, alkyl, hydroxy, methoxy, halo, alkoxy, trifluoromethyl, nitro, amino, aminocarbonyl, aminosulfonyl;
(c) R2 and R3 can be taken together to form a 5 or 6 member aromatic or non-aromatic ring, which can contain from 0 to 3 heteroatoms selected from the group of N, O, or S;
(d) R₄ is hydrogen, alkyl, halo, alkoxy, perfluoroalkyl, perfluoroalkoxy, or nitro;
(e) R₃ and R₄ when taken together can form a 5 or 6 membered ring and can contain one or more heteroatoms; and
(f) n equals 1 or 2.

Preferably, the aryl piperazine moiety comprises one or more of the following substitutions:
(i) R₄ is alkyl, halo, alkoxy, or perfluoroalkyl;
(ii) R₃ and R₄ when taken together are either a methylenedioxy or ethylenedioxy group.

Generally, any moiety A can be combined with any linker L and any moiety B to produce a composite compound according to the present invention. However, in one embodiment the composite compounds of the present invention include, but are not limited to, the following structure:

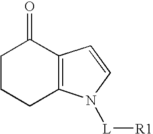

(a) wherein L is as described below; and
(b) wherein R1 is:

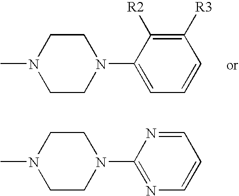

and R2 and R3 are the same or independently hydrogen, alkyl, hydroxy, methoxy, halo, alkoxy, trifluoromethyl, nitro, amino, aminocarbonyl, or aminosulfonyl.

### Linker Moiety

The linker moiety (L) used in the present compounds can be a straight chain alkyl group of the formula —(CH₂)ₘ—, where m is an integer from 1 to 6 and more preferably either 3, 4, or 5. Alternatively, the linker can be an alkyl substituted hydrocarbyl moiety of the following formula (IX):

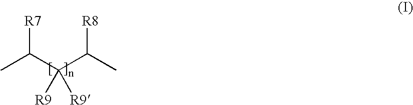

where:
(i) n is 0, 1 or 2;
(ii) R7 and R8 are hydrogen, methyl or ethyl;
(iii) R9 and R9′ are both hydrogen, methyl or ethyl;
(iv) if n is 1 and R7 or R8 is methyl or ethyl, then R9 and R9′ are hydrogen;
(v) if n is 1 and R7 and R8 are hydrogen, then R9 and R9′ are methyl or ethyl; and
(vi) if n is 2, then R9 and R9′ are hydrogen and one or both of R7 and R8 are methyl or ethyl.

The linker moiety can modulate properties of the present compounds. For example, a straight chain alkyl linker comprising two carbon atoms would provide a more rigid linkage than a longer alkyl linker. Such rigidity can produce greater specificity in target binding, while a less rigid linker moiety can produce greater potency. The solubility characteristics of the present compounds can also be affected by the nature of the linker moiety.

The use of a linker according to formula (IX) above is believed to provide a more rigid linkage compared to a straight chain linker moiety with the same number of carbon atoms in the chain. This allows for further control over the properties of the present compounds.

In another embodiment, linker moiety (L) can be a phenyl or a benzyl linked to a hydrocarbyl chain by group Y where group Y is located on the meta or para positions of the aromatic ring. Group Y can be nothing such that the hydrocarbyl chain is directly linked to the phenyl group. Group Y can also be an ether, thioether, carbonyl, thiocarbonyl, carboxamido, aminocarbonyl, amino, oxycarbonylamino, aminocarbonyloxy, aminocarbonylamino, oxythiocarbonylamino, aminothiocarbonyloxy, aminothiocarbonylamino, aminosulfonyl, or sulfonamido group.

The compounds of the present invention further include, but are not limited to, the following compounds:

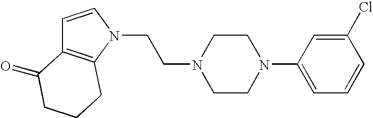

1-{2-[4-(3-Chlorophenyl)piperazin-1-yl]ethyl}-1,5,6,7-tetrahydroindol-4-one (Compound A);

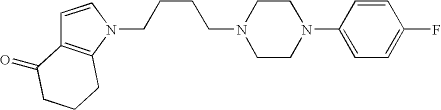

1-{4-[4-(4-Fluorophenyl)piperazin-1-yl]butyl}-1,5,6,7-tetrahydroindol-4-one (Compound B);

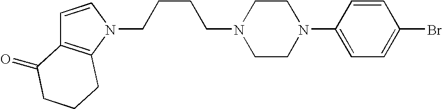

1-{4-[4-(4-Bromophenyl)piperazin-1-yl]butyl}-1,5,6,7-tetrahydroindol-4-one (Compound C);

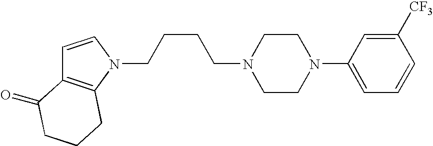

1-{4-[4-(3-Trifluoromethylphenyl)piperazin-1-yl]butyl}-1,5,6,7-tetrahydroindol-4-one (Compound D);

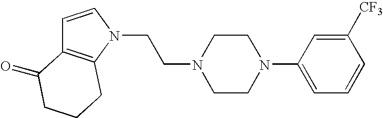

1-{2-[4-(3-Trifluoromethylphenyl)piperazin-1-yl]ethyl}-1,5,6,7-tetrahydroindol-4-one (Compound E);

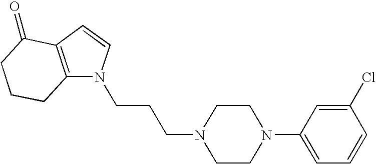

1-{3-[4-(3-Chlorophenyl)piperazin-1-yl]propyl}-1,5,6,7-tetrahydroindol-4-one (Compound F); and

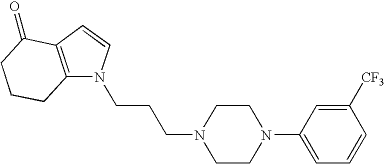

1-{3-[4-(3-Trifluoromethylphenyl)piperazin-1-yl]propyl}-1,5,6,7-tetrahydroindol-4-one (Compound G).

Table 1 below lists further specific embodiments of the present compounds.

TABLE 1

| | |
|---|---|
| 1 | 1-{2-[4-(4-Fluorophenyl)piperazin-1-yl]ethyl}-1,5,6,7-tetrahydroindol-4-one |
| 2 | 1-{3-[4-(4-Fluorophenyl)piperazin-1-yl]propyl}-1,5,6,7-tetrahydroindol-4-one |
| 3 | 1-{5-[4-(4-Fluorophenyl)piperazin-1-yl]pentyl}-1,5,6,7-tetrahydroindol-4-one |
| 4 | 1-{2-[4-(4-Chlorophenyl)piperazin-1-yl]ethyl}-1,5,6,7-tetrahydroindol-4-one |
| 5 | 1-{3-[4-(4-Chlorophenyl)piperazin-1-yl]propyl}-1,5,6,7-tetrahydroindol-4-one |
| 6 | 1-{4-[4-(4-Chlorophenyl)piperazin-1-yl]butyl}-1,5,6,7-tetrahydroindol-4-one |
| 7 | 1-{2-[4-(4-Methoxyphenyl)piperazin-1-yl]ethyl}-1,5,6,7-tetrahydroindol-4-one |
| 8 | 1-{3-[4-(4-Methoxyphenyl)piperazin-1-yl]propyl}-1,5,6,7-tetrahydroindol-4-one |
| 9 | 1-{4-[4-(4-Methoxyphenyl)piperazin-1-yl]butyl}-1,5,6,7-tetrahydroindol-4-one |
| 10 | 1-{2-[4-(2-Fluorophenyl)piperazin-1-yl]ethyl}-1,5,6,7-tetrahydroindol-4-one |
| 11 | 1-{3-[4-(2-Fluorophenyl)piperazin-1-yl]propyl}-1,5,6,7-tetrahydroindol-4-one |
| 12 | 1-{4-[4-(2-Fluorophenyl)piperazin-1-yl]butyl}-1,5,6,7-tetrahydroindol-4-one |
| 13 | 1-{2-[4-(4-Trifluoromethylphenyl)piperazin-1-yl]ethyl}-1,5,6,7-tetrahydroindol-4-one |
| 14 | 1-{3-[4-(4-Trifluoromethylphenyl)piperazin-1-yl]propyl}-1,5,6,7-tetrahydroindol-4-one |
| 15 | 1-{4-[4-(4-Trifluoromethylphenyl)piperazin-1-yl]butyl}-1,5,6,7-tetrahydroindol-4-one |
| 16 | 1-{2-[4-(4-Bromophenyl)piperazin-1-yl]ethyl}-1,5,6,7-tetrahydroindol-4-one |
| 17 | 1-{3-[4-(4-Bromophenyl)piperazin-1-yl]propyl}-1,5,6,7-tetrahydroindol-4-one |
| 18 | 1-{5-[4-(4-Bromophenyl)piperazin-1-yl]pentyl}-1,5,6,7-tetrahydroindol-4-one |
| 19 | 1-{2-[4-p-Tolylpiperazin-1-yl]ethyl}-1,5,6,7-tetrahydroindol-4-one |
| 20 | 1-{3-[4-p-Tolylpiperazin-1-yl]propyl}-1,5,6,7-tetrahydroindol-4-one |
| 21 | 1-{4-[4-p-Tolylpiperazin-1-yl]butyl}-1,5,6,7-tetrahydroindol-4-one |
| 22 | 1-{2-[4-(2,3-Dimethylphenyl)piperazin-1-yl]ethyl}-1,5,6,7-tetrahydroindol-4-one |
| 23 | 1-{3-[4-(2,3-Dimethylphenyl)piperazin-1-yl]propyl}-1,5,6,7-tetrahydroindol-4-one |
| 24 | 1-{4-[4-(2,3-Dimethylphenyl)piperazin-1-yl]butyl}-1,5,6,7-tetrahydroindol-4-one |
| 25 | 1-{2-[4-(3,4-Dichlorophenyl)piperazin-1-yl]ethyl}-1,5,6,7-tetrahydroindol-4-one |
| 26 | 1-{3-[4-(3,4-Dichlorophenyl)piperazin-1-yl]propyl}-1,5,6,7-tetrahydroindol-4-one |
| 27 | 1-{4-[4-(3,4-Dichlorophenyl)piperazin-1-yl]butyl}-1,5,6,7-tetrahydroindol-4-one |
| 28 | 1-{2-[4-(3,4-Difluorophenyl)piperazin-1-yl]ethyl}-1,5,6,7-tetrahydroindol-4-one |
| 29 | 1-{3-[4-(3,4-Difluorophenyl)piperazin-1-yl]propyl}-1,5,6,7-tetrahydroindol-4-one |
| 30 | 1-{4-[4-(3,4-Difluorophenyl)piperazin-1-yl]butyl}-1,5,6,7-tetrahydroindol-4-one |
| 31 | 1-{2-[4-(3,4-Dimethylphenyl)piperazin-1-yl]ethyl}-1,5,6,7-tetrahydroindol-4-one |
| 32 | 1-{3-[4-(3,4-Dimethylphenyl)piperazin-1-yl]propyl}-1,5,6,7-tetrahydroindol-4-one |
| 33 | 1-{4-[4-(3,4-Dimethylphenyl)piperazin-1-yl]butyl}-1,5,6,7-tetrahydroindol-4-one |
| 34 | 1-{2-[4-(2,3-Dichlorophenyl)piperazin-1-yl]ethyl}-1,5,6,7-tetrahydroindol-4-one |
| 35 | 1-{3-[4-(2,3-Dichlorophenyl)piperazin-1-yl]propyl}-1,5,6,7-tetrahydroindol-4-one |
| 36 | 1-{4-[4-(2,3-Dichlorophenyl)piperazin-1-yl]butyl}-1,5,6,7-tetrahydroindol-4-one |
| 37 | 1-[2-(4-(2-Naphthyl)piperazin-1-yl)ethyl]-1,5,6,7-tetrahydroindol-4-one |
| 38 | 1-[3-(4-(2-Naphthyl)piperazin-1-yl)propyl]-1,5,6,7-tetrahydroindol-4-one |
| 39 | 1-[4-(4-(2-Naphthyl)piperazin-1-yl)butyl]-1,5,6,7-tetrahydroindol-4-one |
| 40 | 1-{2-[4-(2,3-Dihydrobenzo[1,4]dioxin-6-yl)piperazin-1-yl]ethyl}-1,5,6,7-tetrahydroindol-4-one |
| 41 | 1-{3-[4-(2,3-Dihydrobenzo[1,4]dioxin-6-yl)piperazin-1-yl]propyl}-1,5,6,7-tetrahydroindol-4-one |
| 42 | 1-{4-[4-(2,3-Dihydrobenzo[1,4]dioxin-6-yl)piperazin-1-yl]butyl}-1,5,6,7-tetrahydroindol-4-one |
| 43 | 1-{2-[4-(2,4-Dichlorophenyl)piperazin-1-yl]ethyl}-1,5,6,7-tetrahydroindol-4-one |
| 44 | 1-{3-[4-(2,4-Dichlorophenyl)piperazin-1-yl]propyl}-1,5,6,7-tetrahydroindol-4-one |
| 45 | 1-{4-[4-(2,4-Dichlorophenyl)piperazin-1-yl]butyl}-1,5,6,7-tetrahydroindol-4-one |
| 46 | 1-{2-[4-(2,4-Difluorophenyl)piperazin-1-yl]ethyl}-1,5,6,7-tetrahydroindol-4-one |
| 47 | 1-{3-[4-(2,4-Difluorophenyl)piperazin-1-yl]propyl}-1,5,6,7-tetrahydroindol-4-one |
| 48 | 1-{4-[4-(2,4-Difluorophenyl)piperazin-1-yl]butyl}-1,5,6,7-tetrahydroindol-4-one |
| 49 | 1-{2-[4-(2,4-Dimethylphenyl)piperazin-1-yl]ethyl}-1,5,6,7-tetrahydroindol-4-one |
| 50 | 1-{3-[4-(2,4-Dimethylphenyl)piperazin-1-yl]propyl}-1,5,6,7-tetrahydroindol-4-one |
| 51 | 1-{4-[4-(2,4-Dimethylphenyl)piperazin-1-yl]butyl}-1,5,6,7-tetrahydroindol-4-one |
| 52 | 1-{2-[4-(5-Bromopyrimidin-2-yl)piperazin-1-yl]ethyl}-1,5,6,7-tetrahydroindol-4-one |
| 53 | 1-{3-[4-(5-Bromopyrimidin-2-yl)piperazin-1-yl]propyl}-1,5,6,7-tetrahydroindol-4-one |
| 54 | 1-{4-[4-(5-Bromopyrimidin-2-yl)piperazin-1-yl]butyl}-1,5,6,7-tetrahydroindol-4-one |
| 55 | 1-{2-[4-(2,3,4-Trichlorophenyl)piperazin-1-yl]ethyl}-1,5,6,7-tetrahydroindol-4-one |
| 56 | 1-{3-[4-(2,3,4-Trichlorophenyl)piperazin-1-yl]propyl}-1,5,6,7-tetrahydroindol-4-one |
| 57 | 1-{4-[4-(2,3,4-Trichlorophenyl)piperazin-1-yl]butyl}-1,5,6,7-tetrahydroindol-4-one |
| 58 | 1-{2-[4-(2,3,4-Trifluorophenyl)piperazin-1-yl]ethyl}-1,5,6,7-tetrahydroindol-4-one |
| 59 | 1-{3-[4-(2,3,4-Trifluorophenyl)piperazin-1-yl]propyl}-1,5,6,7-tetrahydroindol-4-one |
| 60 | 1-{4-[4-(2,3,4-Trifluorophenyl)piperazin-1-yl]butyl}-1,5,6,7-tetrahydroindol-4-one |
| 61 | 1-{2-[4-(3-Chloro-4-fluorophenyl)piperazin-1-yl]ethyl}-1,5,6,7-tetrahydroindol-4-one |
| 62 | 1-{3-[4-(3-Chloro-4-fluorophenyl)piperazin-1-yl]propyl}-1,5,6,7-tetrahydroindol-4-one |
| 63 | 1-{4-[4-(3-Chloro-4-fluorophenyl)piperazin-1-yl]butyl}-1,5,6,7-tetrahydroindol-4-one |
| 64 | 1-{5-[4-(3-Chloro-4-fluorophenyl)piperazin-1-yl]pentyl}-1,5,6,7-tetrahydroindol-4-one |
| 65 | 1-{2-[4-(4-Fluoro-3-trifluoromethylphenyl)piperazin-1-yl]ethyl}-1,5,6,7-tetrahydroindol-4-one |
| 66 | 1-{3-[4-(4-Fluoro-3-trifluoromethylphenyl)piperazin-1-yl]propyl}-1,5,6,7-tetrahydroindol-4-one |
| 67 | 1-{4-[4-(4-Fluoro-3-trifluoromethylphenyl)piperazin-1-yl]butyl}-1,5,6,7-tetrahydroindol-4-one |
| 68 | 1-{2-[4-(4-Chloro-2-methoxyphenyl)piperazin-1-yl]ethyl}-1,5,6,7-tetrahydroindol-4-one |
| 69 | 1-{3-[4-(4-Chloro-2-methoxyphenyl)piperazin-1-yl]propyl}-1,5,6,7-tetrahydroindol-4-one |
| 70 | 1-{4-[4-(4-Chloro-2-methoxyphenyl)piperazin-1-yl]butyl}-1,5,6,7-tetrahydroindol-4-one |
| 71 | 1-{2-[4-(4-Chloro-3-trifluoromethylphenyl)piperazin-1-yl]ethyl}-1,5,6,7-tetrahydroindol-4-one |
| 72 | 1-{3-[4-(4-Chloro-3-trifluoromethylphenyl)piperazin-1-yl]propyl}-1,5,6,7-tetrahydroindol-4-one |
| 73 | 1-{4-[4-(4-Chloro-3-trifluoromethylphenyl)piperazin-1-yl]butyl}-1,5,6,7-tetrahydroindol-4-one |
| 74 | 1-{5-[4-(4-Chloro-3-trifluoromethylphenyl)piperazin-1-yl]pentyl}-1,5,6,7-tetrahydroindol-4-one |
| 75 | 1-{2-[4-(6-Chloroquinolin-4-yl)piperazin-1-yl]ethyl}-1,5,6,7-tetrahydroindol-4-one |
| 76 | 1-{3-[4-(6-Chloroquinolin-4-yl)piperazin-1-yl]propyl}-1,5,6,7-tetrahydroindol-4-one |
| 77 | 1-{4-[4-(6-Chloroquinolin-4-yl)piperazin-1-yl]butyl}-1,5,6,7-tetrahydroindol-4-one |
| 78 | 1-{2-[4-(Thieno[3,2-d]pyrimidin-4-yl)piperazin-1-yl]ethyl}-1,5,6,7-tetrahydroindol-4-one |
| 79 | 1-{3-[4-(Thieno[3,2-d]pyrimidin-4-yl)piperazin-1-yl]propyl}-1,5,6,7-tetrahydroindol-4-one |
| 80 | 1-{4-[4-(Thieno[3,2-d]pyrimidin-4-yl)piperazin-1-yl]butyl}-1,5,6,7-tetrahydroindol-4-one |
| 81 | 1-{2-[4-(4-Chloronaphthalen-1-yl)piperazin-1-yl]ethyl}-1,5,6,7-tetrahydroindol-4-one |
| 82 | 1-{3-[4-(4-Chloronaphthalen-1-yl)piperazin-1-yl]propyl}-1,5,6,7-tetrahydroindol-4-one |
| 83 | 1-{4-[4-(4-Chloronaphthalen-1-yl)piperazin-1-yl]butyl}-1,5,6,7-tetrahydroindol-4-one |
| 84 | 1-{2-[4-(Furo[3,2-c]pyridine-4-yl)piperazin-1-yl]ethyl}-1,5,6,7-tetrahydroindol-4-one |
| 85 | 1-{3-[4-(Furo[3,2-c]pyridine-4-yl)piperazin-1-yl]propyl}-1,5,6,7-tetrahydroindol-4-one |
| 86 | 1-{4-[4-(Furo[3,2-c]pyridine-4-yl)piperazin-1-yl]butyl}-1,5,6,7-tetrahydroindol-4-one |
| 87 | 1-{2-[4-(4-Chloro-2-fluorophenyl)piperazin-1-yl]ethyl}-1,5,6,7-tetrahydroindol-4-one |
| 88 | 1-{3-[4-(4-Chloro-2-fluorophenyl)piperazin-1-yl]propyl}-1,5,6,7-tetrahydroindol-4-one |
| 89 | 1-{4-[4-(4-Chloro-2-fluorophenyl)piperazin-1-yl]butyl}-1,5,6,7-tetrahydroindol-4-one |
| 90 | 1-{4-[4-(2,3-Dichlorophenyl)piperazin-1-yl]butyl}-1,5,6,7-tetrahydroindol-4-one |
| 91 | 1-{3-[4-(2,3-Dichlorophenyl)piperazin-1-yl]propyl}-1,5,6,7-tetrahydroindol-4-one |
| 92 | 1-{2-[4-(2,3-Dichlorophenyl)piperazin-1-yl]ethyl}-1,5,6,7-tetrahydroindol-4-one |
| 93 | 1-{4-[4-(2,3-Dimethylphenyl)piperazin-1-yl]butyl}-1,5,6,7-tetrahydroindol-4-one |
| 94 | 1-{3-[4-(2,3-Dimethylphenyl)piperazin-1-yl]propyl}-1,5,6,7-tetrahydroindol-4-one |
| 95 | 1-{2-[4-(2,3-Dimethylphenyl)piperazin-1-yl]ethyl}-1,5,6,7-tetrahydroindol-4-one |

### Compound Properties

Preferred compounds have a logP of from about 1 to about 4 to enhance bioavailability and, when desired, central nervous system (CNS) penetration. Using this guideline, one of ordinary skill in the art can choose the appropriate arylpiperazine moieties to use in combination with a particular A moiety in order to ensure the bioavailability and CNS penetration of a compound of the present invention. For example, if a highly hydrophobic A moiety is chosen, with particularly hydrophobic substituents, then a more hydrophilic arylpiperazine moiety can be used.

A number of the present compounds are optically active, owing to the presence of chiral carbons or other centers of asymmetry. All of the possible enantiomers or diastereoisomers of such compounds are included herein unless otherwise indicated despite possible differences in activity.

In general, the present compounds also include salts and prodrug esters of the compounds described herein. It is well known that organic compounds, including substituted tetrahydroindolones, arylpiperazines and other components of the present compounds, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound in the present application includes such salt forms as occur at physiological pH or at the pH of a pharmaceutical composition unless specifically excluded.

Similarly, prodrug esters can be formed by reaction of either a carboxyl or a hydroxyl group on the compound with either an acid or an alcohol to form an ester. Typically, the acid or alcohol includes an alkyl group such as methyl, ethyl, propyl, isopropyl, butyl, isobutyl, and tertiary butyl. These groups can be substituted with substituents such as hydroxy, halo, or other substituents. Such prodrugs are well known in the art. The prodrug is converted into the active compound by hydrolysis of the ester linkage, typically by intracellular enzymes. Other suitable groups that can be used to form prodrug esters are well known in the art.

### SYNTHESIS EXAMPLES

The following representative methods for synthesizing exemplary compounds used in the present methods are intended as examples. Persons having ordinary skill in the art of medicinal and/or organic chemistry will understand that other starting materials, intermediates, and reaction conditions are possible. Furthermore, it is understood that various salts and esters of these compounds can be made and that these salts and esters can have a biological activity similar or equivalent to the parent compound. Generally, such salts have halides or organic acids as anion counterions. However, other anions can also be used and are considered within the scope of the present invention.

### Example 1

### Synthesis of 1-{2-[4-(3-Trifluoromethylphenyl)piperazin-1-yl]ethyl}-1,5,6,-7-tetrahydroindol-4-one

This example demonstrates a method of preparing 1-{2-[4-(3

Trifluoromethylphenyl)piperazin-1-yl]ethyl}-1,5,6,7-tetrahydroindol-4-one by a two step procedure. Generally, the arylpiperazine moieties are prepared first, then the arylpiperazine molecules are reacted with tetrahydroindolones.

### Step 1: Preparation of 1-(2-Chloroethyl)-4-(3-trifluoromethylphenyl)piperazine

To a 100 mL flask was added 4-(3-trifluoromethylphenyl)piperazine HCl (5035 mg, 18.88 mmol) and 60 mL dichloromethane. 1-Bromo-2-chloroethane (1730 μL, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(2-chloroethyl)-4-(3-trifluoromethylphenyl)piperazine.

### Step 2: Preparation of 1-{2-[4-(3-Trifluoromethylphenyl)piperazin-1-yl]ethyl}-1,5,6,7-tetrahydroindol-4-one

Sodium hydride (60% in oil) (85 mg, 2.1 mmol, 1.8 eq.) was added to a 10 mL pear-shaped flask. The solid was rinsed twice with 2 mL hexane to remove oil, then 3 mL anhydrous N,N-dimethylformamide (DMF) was added. 1,5,6,7-Tetrahydroindol-4-one (186.7 mg, 1.38 mmol, 1.159 eq.) was added slowly, with stirring and hydrogen evolved. The walls of the flask were washed with an additional 1 mL of anhydrous DMF. 1-(2-Chloroethyl)-4-(3-trifluoromethylphenyl)piperazine (349.00 mg, 1.19 mmol, 1.000 eq) was added as a solution in 2 mL DMF, and the mixture was stirred under nitrogen at 25 C for 8 hours. The resulting mixture was acidified with 1N HC1 to pH 6, and extracted with dichloromethane. The organic layer was washed four times with 25 mL water, dried over sodium sulfate and concentrated in vacuo to an oil which was purified by column chromatography using 5% methanol in dichloromethane as eluant resulting in the title compound as an oil. The oil was dissolved in 5 mL of 50% dichloromethane in hexanes. A solution of 4N HCl in dioxane (200 μL) was added and the mixture stirred for 30 minutes followed by vacuum filtration of the suspension. A white powder of the product HCl salt was recovered.

### Example 2

### Synthesis of 1-{3-[4-(3-Trifluoromethylphenyl)piperazin-1-yl]propyl}-1,5,6,7-tetrahydroindol-4-one

### Step 1: Preparation of 1-(3-Chloropropyl)-4-(3-trifluoromethylphenyl)piperazine

To a 100 mL flask was added 1-(3-trifluoromethylphenyl)piperazine HCl (5035 mg, 18.88 mmol) and 60 mL dichloromethane. 1-Bromo-3-chloropropane (1730 CL, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(3-chloropropyl)-4-(3-trifluoromethylphenyl)piperazine.

### Step 2: Preparation of 1-{2-[4-(3-Trifluoromethylphenyl)piperazin-1-yl]propyl}-1,5,6,7-tetrahydroindol-4-one

The compound is synthesized by reacting the 1-(3-chloropropyl)-4-(3-trifluoromethylphenyl)piperazine with 1,5,6,7-tetrahydroindol-4-one using step 2 of Example 1.

### Example 3

### Synthesis of 1-{3-[4-(3-Chlorophenyl)piperazine-1-yl]propyl}-1,5,6,7-tetrahydroindol-4-one

Since 1-(3-Chloropropyl)-4-(3-chlorophenyl)piperazine HCl is commercially available, step one was omitted.

To a solution of 1,5,6,7-tetrahydroindol-4-one (135 mg, 1.0 mmol) in 5 mL dimethylsulfoxide was added powdered sodium hydroxide (84 mg, 2.1 mmol) and the solution stirred for 15 minutes at 25° C. 1-(3-Chloropropyl)-4-(3-chlorophenyl)piperazine HCl (310 mg, 1.0 mmol) was then added and stirring continued overnight. Upon completion, by thin-layer chromatography (TLC), the reaction was partitioned between 50 mL each of dichloromethane and water then separated. The water layer was extracted with 50 mL more of dichloromethane and the combined organic layers washed with brine, dried with sodium sulfate, and concentrated in vacuo to dryness. The crude product was purified via flash chromatography eluting with an ethyl acetate and dichloromethane mixture resulting in the title compound as an oil. The oil was dissolved in 5 mL of 50% dichloromethane in hexanes. A solution of 4N HCl in dioxane (200 □L) was added and the mixture stirred for 30 minutes followed by vacuum filtration of the suspension. A white powder of the product HCl salt was recovered.

### Example 4

### Synthesis of 1-{3-[4-(2-Methoxyphenyl)piperazine-1-yl]propyl}-1,5,6,7-tetrahydroindol-4-one

### Step 1: Preparation of 1-(3-Chloropropyl)-4-(2-methoxyphenyl)piperazine

The 1-(3-Chloropropyl)-4-(3-trifluoromethylphenyl)piperazine is prepared by the same method as disclosed in step 1 of example 2 employing 1-(2-Methoxyphenyl)piperazine HCl instead.

### Step 2: Preparation of 1-{3-[4-(2-Methoxyphenyl)piperazine-1-yl]propyl}-1,5,6,7-tetrahydroindol-4-one

The compound is prepared by the same method as disclosed in step 2 of example 3.

### Example 5

### Synthesis of 1-{3-[4-(2-Pyrimidyl)piperazine-1-yl]propyl}-1,5,6,7-tetrahydroindol-4-one

### Step 1: Preparation of 1-(3-Chloropropyl)-4-(2-pyrimidyl)piperazine

The compound is prepared by the same method as disclosed in step 1 of example 2 employing 1-(2-Pyrimidyl)piperazine.2HCl instead.

### Step 2: Preparation of 1-{3-[4-(2-Pyrimidyl)piperazine-1-yl]propyl}-1,5,6,7-tetrahydroindol-4-one

The compound is prepared by the same method as disclosed in step 2 of Example 3.

### Example 6

### Synthesis of 1-{2-[4-(3-Chlorophenyl)piperazin-1-yl]ethyl}-1,5,6,7-tetrahydroindol-4-one (Compound A)

### Step 1: Preparation of 1-(2-Chloroethyl)-4-(3-chlorophenyl)piperazine

A mixture of (3-chlorophenyl)piperazine HCl (51.5 mmol) and powdered sodium hydroxide (103 mmol) in DMSO (75 mL) was treated with 2-bromo-1-chloroethane (77.2 mmol) and stirred at ambient temperature for 4 hours. The reaction was poured into ice cold water (200 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate, filtered and the solvent removed under vacuum. Flash chromatography (chloroform:acetone 50:1 to 20:1) yielded an oil (7.95 g) as the titled compound.

### Step 2: 1-{2-[4-(3-Chlorophenyl)piperazin-1-yl]ethyl}-1,5,6,7-tetrahydroindol 4-one

To a solution of 1,5,6,7-tetrahyroindol-4-one (51.5 mmol) in DMSO (60 mL) was added powdered sodium hydroxide (53.9 mmol) and the mixture was stirred at ambient temperature for 0.5 hours. 1-(2-chloroethyl)-4-(3-chlorophenyl)piperazine (49.0 mmol) was then added as a solution in DMSO (20 mL) and the resulting mixture stirred at ambient temperature for 24 hours then heated to approximately 60° C. for 2 hours, after which time TLC (ethyl acetate:dichloromethane 1:1) showed complete reaction. The reaction was poured into ice cold water (300 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate and the solvent removed under vacuum. The resulting sludge was triturated with hexanes (100 mL) for 2 hours and the suspension vacuum filtered and washed with hexanes. The obtained solid was dried under vacuum resulting in a tan powder (14.57 g) as the titled compound.

### Example 7

### Synthesis of 1-{2-[4-(2-Methoxyphenyl)piperazin-1-yl]ethyl}-1,5,6,7-tetrahydroindol-4-one

### Step 1: Preparation of 1-(2-Chloroethyl)-4-(2-methoxyphenyl)piperazine

A mixture of 1-(2-methoxyphenyl)piperazine HCl (52.5 mmol) and powdered sodium hydroxide (105 mmol) in DMSO (40 mL), was stirred at ambient temperature. After 0.5 hours, 1-bromo-2-chloroethane (78.8 mmol) was added to the solution and left to stir for 4 hours. The reaction was monitored by TLC (ethyl acetate: dichloromethane 1:4), upon completion, the mixture was poured into 200 mL of ice water and the product was extracted with dichloromethane twice, dried with sodium sulfate, and solvent was removed under vacuum. Flash chromatography (ethyl acetate: dichloromethane, 1:5 yielded an oil of the title compound (7.30 g).

### Step 2: Preparation of 1-{2-[4-(2-Methoxyphenyl)piperazin-1-yl]ethyl}-1,5,6,7-tetrahydroindol-4-one

A mixture of 1,5,6,7-tetrahyroindol-4-one (30.1 mmol) and powdered sodium hydroxide (31.6 mmol) in DMSO (15 mL) was heated for 0.5 h, and then treated with a solution of 1-(2-chloroethyl)-4-(2-methoxyphenyl)piperazine (7.30 g) in DMSO (30 mL) dropwise. The reaction was left under heat and was monitored by TLC (ethyl acetate: dichloromethane, 1:1). After completion (˜8 hours), the reaction mixture was poured into ice water (300 mL) and extracted with dichloromethane twice, dried with sodium sulfate and the solvent removed under vacuum. Flash chromatography (ethyl acetate: dichloromethane, 1:4) yielded an oil, (7.25 g).

### Example 8

### Synthesis of 1-{4-[4-(3-Trifluoromethylphenyl)piperazin-1-yl]butyl}-1,5,6,-7-tetrahydroindol-4-one

### Step 1: Synthesis of 1-(4-Chlorobutyl)-1,5,6,7-tetrahydroindol-4-one

To a solution of 1,5,6,7-tetrahydroindol-4-one (10.0 g, 74.0 mmol) in acetone (300 mL) was added powdered sodium hydroxide (3.26 g, 81.4 mmol) and the mixture stirred at ambient temperature for 0.25 hours. 1-Bromo-4-chlorobutane (9.38 mL, 81.4 mmol) was then added and the resulting mixture stirred at ambient temperature for 7 hours after which time TLC (ethyl acetate:dichloromethane 1:1) showed complete reaction. The reaction was gravity filtered to remove salts, and the filtrate concentrated to dryness under vacuum. The resulting residue was dissolved in dichloromethane (200 mL) and gravity filtered again to remove more salts. The filtrate was then washed with water, dried with sodium sulfate, filtered and the solvent removed under vacuum to yield an oil. Flash chromatography using 6 in. of silica gel in a 5.5 cm column eluting with 1:1 followed by 2:1 ethyl acetate:hexane on half of the residue yielded 9.0 g of an oil which contained ˜6.0 g of pure product (72%) and ˜3.0 g of acetone aldol condensation product (4-hydroxy-4-methyl-2-pentanone). The oil was taken to the next step without further purification.

### Step 2: Synthesis of 1-{4-[4-(3-Trifluoromethylphenyl)piperazin-1-yl]butyl}-1,5,6,7-tetrahydroindol-4-one

A mixture of 1-(4-Chlorobutyl)-1,5,6,7-tetrahydroindol-4-one (6.0 g, 26.6 mmol, as a mixture with 3.0 g of 4-hydroxy-4-methyl-2-pentanone) and sodium iodide (4.38 g, 29.2 mmol) in acetonitrile (100 mL) was heated at reflux for 6 hours. (3-Trifluoromethylphenyl)piperazine (5.81 g, 25.2 mmol) and potassium carbonate (3.67 g, 26.6 mmol) was then added and reflux continued for 16 hours. TLC (ethyl acetate:dichloromethane 1:1) showed complete reaction. The reaction was poured into ice cold water (400 mL) and stirred for 0.5 hours. An oil separated out and was isolated from the mixture. The oil was dissolved with dichloromethane (150 mL), washed with water and brine, then dried with sodium sulfate, filtered and the solvent removed under vacuum to yield the title compound as an oil (9.7 g, 91.5%).

Preparation of Oxalate salt of 1-{4-[4-(3-Trifluoromethylphenyl)piperazin-1-yl]butyl}-1,5,6,7-tetrahydroindol-4-one. Dissolved compound (4.2 g) in hot ethyl acetate (150 mL), filtered solution hot to remove undissolved solid, and added a solution of oxalic acid (1.08 g, 1.2 eq) in methanol (10 mL) with stirring. A white precipitate formed immediately and the mixture was stirred for 0.5 hours to room temperature. Vacuum filtration and washing with ethyl acetate afforded an off-white powder upon drying (5.0 g, 98%). HPLC Purity was 98.9%.

### Example 9

### Synthesis of 1-{2-[4-(3,4-Dichlorophenyl)piperazin-1-yl]ethyl}-1,5,6,7-tetrahydroindol-4-one

### Step 1: Preparation of 1-(2-Chloroethyl)-4-(3,4-dichlorophenyl)piperazine

A mixture of (3,4-dichlorophenyl)piperazine (500 mg) and powdered sodium hydroxide (87 mg) in DMSO (5 mL) was treated with 2-bromo-1-chloroethane (387 mg) and stirred at ambient temperature for 16 hours. The reaction was poured into ice cold water (15 mL) and stirred for 0.5 hours. A solid mass formed and was separated by decanting the water. The aqueous layer was extracted with dichloromethane (5 mL). The solid mass was dissolved with dichloromethane (5 mL) and the combined organics were dried with sodium sulfate, filtered and the solvent removed under vacuum. Flash chromatography (dichloromethane:methanol 1:0 to 10:1) yielded an oil (230 mg) as the titled compound.

### Step 2: 1-{2-[4-(3,4-Dichlorophenyl)piperazin-1-yl]ethyl}-1,5,6,7-tetrahydroindol-4-one

To a solution of 1,5,6,7-tetrahyroindol-4-one (107 mg) in DMSO (2 mL) was added powdered sodium hydroxide (33 mg) and the mixture was stirred at ambient temperature for 0.5 hours. 1-(2-Chloroethyl)-4-(3,4-dichlorophenyl)piperazine (220 mg) from step 1 was then added as a solution in DMSO (2 mL) and the resulting mixture stirred at ambient temperature for 24 hours then heated to approximately 60° C. for 2 hours, after which time thin layer chromatography (TLC) (ethyl acetate:dichloromethane 1:1) showed complete reaction. The reaction was poured into ice cold water (15 mL) and stirred for 0.5 hours. A solid mass formed and was separated by decanting the water. The aqueous layer was extracted with dichloromethane (10 mL). The solid mass was dissolved with dichloromethane (5 mL) and the combined organics were dried with sodium sulfate and the solvent removed under vacuum to obtain an oil (250 mg) as the titled compound.

### Step 3: Preparation of Oxalate salt of 1-{2-[4-(3,4-Dichlorophenyl)piperazin-1-yl]ethyl}-1,5,6,7-tetrahydroindol 4-one

The compound from step 2 (250 mg) was dissolved in ethyl acetate (5 mL) using heat if required, and a solution of oxalic acid (57 mg) in acetone (0.5 mL) was added with stirring. A precipitate formed immediately and the mixture was stirred for 0.5 hours at room temperature. Vacuum filtration and washing with ethyl acetate afforded an off-white powder upon drying (220 mg).

The same 3-step procedure is used for all ethyl and propyl linkers.

### Example 10

### Synthesis of 1-{4-[4-(3,4-Dichlorophenyl)piperazin-1-yl]butyl}-1,5,6,7-tetrahydroindol-4-one

### Step 1: Synthesis of 1-(4-Chlorobutyl)-1,5,6,7-tetrahydroindol-4-one

To a solution of 1,5,6,7-tetrahydroindol-4-one (10.0 g) in DMSO (100 mL) was added powdered sodium hydroxide (3.26 g) and the mixture was stirred at ambient temperature for 0.25 hours. 1-Bromo-4-chlorobutane (9.38 mL) was then added and the resulting mixture stirred at ambient temperature for 7 hours after which time TLC (ethyl acetate:dichloromethane 1:1) showed complete reaction. The reaction was poured into ice cold water (250 mL) and stirred for 0.5 hours. An oil separated and was isolated with a separatory funnel. The aqueous layer was extracted with dichloromethane (50 mL). The oil was dissolved with dichloromethane (25 mL) and the combined organics were dried with sodium sulfate, filtered and the solvent removed under vacuum. Flash chromatography (ethyl acetate:hexane, 1:1 to 2:1) yielded an oil (6.0 g) as the titled compound.

### Step 2: Synthesis of 1-{4-[4-(3,4-Dichlorophenyl)piperazin-1-yl]butyl}-1,5,6,7-tetrahydroindol-4-one

A mixture of 1-(4-Chlorobutyl)-1,5,6,7-tetrahydroindol-4-one (600 mg) from step 1 and sodium iodide (438 mg) in acetonitrile (10 mL) was heated at reflux for 6 hours. (3,4-Dichlorophenyl)piperazine (581 mg) and potassium carbonate (367 mg) was then added and reflux continued for 16 h. TLC (ethyl acetate:dichloromethane 1:1) showed complete reaction. The reaction was poured into ice cold water (50 mL) and stirred for 0.5 hours. An oil separated out and was isolated from the mixture. The oil was dissolved with dichloromethane (15 mL), washed with water and brine, then dried with sodium sulfate, filtered and the solvent removed under vacuum to yield the title compound as an oil (970 mg).

### Step 3: Oxalate Salt Formation

Oxalate salt formation is done in the same manner as previously described.

The same 3-step procedure is used for all butyl linkers.

### Pharmaceutical Compositions

A pharmaceutical composition can comprise one or more of the present compounds. Such a composition preferably comprises: (1) a therapeutically effective amount of one or more of the present compounds (and/or salts and esters thereof); and (2) a pharmaceutically acceptable excipient.

A pharmaceutically acceptable excipient, including carriers, can be chosen from those generally known in the art including, but not limited to, inert solid diluents, aqueous solutions, or non-toxic organic solvents, depending on the route of administration. If desired, these pharmaceutical formulations can also contain preservatives and stabilizing agents and the like, for example substances such as, but not limited to, pharmaceutically acceptable excipients selected from the group consisting of wetting or emulsifying agents, pH buffering agents, human serum albumin, antioxidants, preservatives, bacteriostatic agents, dextrose, sucrose, trehalose, maltose, lecithin, glycine, sorbic acid, propylene glycol, polyethylene glycol, protamine sulfate, sodium chloride, or potassium chloride, mineral oil, vegetable oils and combinations thereof. Those skilled in the art will appreciate that other carriers also can be used.

Liquid compositions can also contain liquid phase excipients either in addition to or to the exclusion of water. Examples of such additional liquid phases are glycerin, vegetable oils such as cottonseed oil, organic esters such as ethyl oleate, and water-oil emulsions.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. The pharmaceutical compositions of the present invention can be formulated for administration by intravenous infusion, oral, topical, intraperitoneal, intravesical, transdermal, intranasal, rectal, vaginal, intramuscular, intradermal, subcutaneous and intrathecal routes.

Formulations of compound suitable for use in methods according to the present invention can be presented in unit-dose or multi-dose sealed containers, in physical forms such as ampules or vials. The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

### Preclinical Models and Clinical Evaluation

In order to screen for the most effective of the present compounds and pharmaceutical compositions and determine appropriate candidates for further development, as well as to determine appropriate dosages of such compounds and compositions for a human subject, preclinical animal models can be used. Exemplary animal models are set forth below. Preferably, a series of tests is performed in animal models to screen for activity in treating and/or preventing the effects of exposure to nerve agents.

Compounds and compositions are preferably selected using a panel of pre-clinical tests. Preliminary screening tests can be used to determine appropriate dosages to test in follow-on models. Appropriately selected doses of compounds and compositions tested in this way can then be subjected to testing for efficacy against nerve agent exposure.

A. Models for Determining Appropriate Dosages

1. Neuromuscular Coordination Model (Rotarod)

This model can be used to determine the dose of a compound or composition at which unwanted side effects (muscle tone/motor coordination deficits) occur. Animals (C57 Mice) are placed on a rotarod treadmill (model V EE/85, Columbus Instruments, Columbus, Ohio) accelerating from 1 to 80 revolutions/4 minutes. All mice are given two control trials at least 12 hours before oral administration evaluation of compounds. Mice are tested on the rotarod 30 minutes after administration of compounds. The number of seconds each mouse remained on the rotarod is recorded.

Doses at which the coordination of an animal is decreased or at which its motor function is altered, such that the ability of the animal to remain on the rotarod is reduced, are determined. Doses below this are selected for further evaluation.

2. Spontaneous Activity Model (Locomotor Activity)

Ambulatory and non-ambulatory activity can be used to test spontaneous and drug-induced motor activity. The test can be used to profile the potential for a drug to induce hyperactivity or sedation.

In this model, Kinder Scientific photobeam activity monitors are used to record the ambulatory and non-ambulatory motor activity. The monitors track the photobeam breaks made by the animal that are used to calculate the number of ambulatory and fine (non-ambulatory) motor movements. A drug-induced increase in activity can indicate the potential for an adverse event such as hyperactivity. A drug-induced decrease in response can indicate the potential for an adverse event such as sedation. Doses at which no significant change in activity are recorded, and more preferably at which no change in activity are recorded, can be selected for further evaluation.

3. Potentiated Startle (Anxiety Model)

This model can be used to evaluate anxiolytic or anxiogenic effects of a candidate molecule. In this model, Hamilton-Kinder startle chambers can be used for conditioning sessions and for the production and recording of startle responses. A classical conditioning procedure is then used to produce potentiation of startle responses. On the first of 2 days, rats, preferably Long Evans rats, are placed into dark startle chambers having shock grids. Following a 5-minute acclimation period, each rat is administered a 1 mA electric shock (500 ms) preceded by a 5 second presentation of light (15 watt) which remains on for the duration of the shock. Ten presentations of the light and shock are given in each conditioning session.

The rats are then administered a test compound, after which startle testing sessions are conducted. A block of 10 consecutive presentations of acoustic startle stimuli (110 dB, non-light-paired) are presented at the beginning of the session in order to minimize the influences of the initial rapid phase of habituation to the stimulus. This is followed by 20 alternating trials of the noise alone or noise preceded by the light. Excluding the initial trial block, startle response amplitudes for each trial type (noise-alone vs. light+noise) are averaged for each rat across the entire test session.

Compounds and compositions appropriate development preferably do not result in either anxiogenic or anxiolytic activity.

4. Other Models

Other models that can be used to evaluate proper dosages of the present compounds and compositions include the Elevated Plus Maze model, which also evaluates the anxiogenic or anxiolytic activity of a candidate.

B. Evaluation of Prophylactic Protection from Nerve Agent Exposure

Male ICR mice from Charles River (20 to 30 grams average weight) are treated with one of the present compounds i.m. 15 or 60 minutes, or by gavage 30 or 120 minutes, before challenge with a dose of 2xLD50 of soman (LD50=98 μg/kg without atropine, LD50=130 μg/kg with 11.2 mg/kg of atropine). As a negative control, saline is administered instead of a test compound. As a positive control for survival, pyridostigmine (0.1 mg/kg, i.m.or 0.82 mg/kg orally) is administered to a separate group of animals.

All subject animals receive atropine sulfate (11.2 mg/kg) and 2-PAM (25 mg/kg) i.m. exactly 10 seconds after soman challenge, using a total dose volume of 0.5 ml/kg body weight. All animals are then allocated to pretreatment cells in a randomized block design. Groups of ten mice are used in each experiment and survivors in each group are noted after 24 hours. The 24-hour survival of animals pretreated with each dose of one of the present compounds is compared with the 24-hour survival observed in the negative control group. A survival difference of at least four indicates improved efficacy of the candidate compound over that observed with the negative control group.

Once improved efficacy of a candidate compound is shown, the candidate can further be tested for efficacy in the absence of atropine and/or 2-PAM administration. This can lead to the identification of compounds capable of providing at least partial prophylaxis with respect to the effects of organophosphate nerve agent exposure when used as single agents.

In vitro models of neuroprotection can also be used to evaluate candidate compounds. Nerve Growth Factor (NGF) and its cell surface target play a role in neuronal cell differentiation, growth and repair mechanisms and offers neuroprotection in in vitro experiments. The present compounds can be tested as a cytoprotective agent in neuronal cells deprived of growth factor (NGF and serum) for 24 hours.

C. Evaluation of Post-Exposure Protection from Nerve Agents

Male ICR mice from Charles River (20 to 30 grams average weight) are treated with one of the present compounds administered i.m. 10 seconds after challenge with a dose of 2xLD50 of soman or tabun (aqueous solution containing 0.9% NaCl). Compounds are given simultaneously with atropine sulfate (11.2 mg/kg). As a negative control, atropine sulfate (11.2 mg/kg) and 2-PAM (25 mg/kg) are given without a test compound (no mice would be expected to survive). As a positive control for survival, HI-6 (9.6 mg/kg) is administered with atropine sulfate (11.2 mg/kg) to a separate group of animals. All injections are administered i.m. using a dose volume of 0.5 mL/kg body weight.

All animals are allocated to treatment cells in a randomized block design. Groups of ten mice are used in each experiment and survivors in each group are noted after 24 hours. The 24-hour survival of animals injected with each dose of a test compound is compared to the 24-hour survival observed in the negative control group. A survival difference of at least four indicates improved efficacy of the candidate compound over that observed with the negative control group.

D. Further Evaluation of Post-Exposure Protection

The effects produced by the present compounds with respect to the prevention and treatment of nerve agent exposure can be also evaluated through the use of further preclinical testing, as described below. Such testing can be performed, for example, with male FVB/N mice (20-25 grams, available from Harlan Laboratories). This strain develops neurodegeneration following organophosphate (OP) poisoning and expresses fluorojade staining in cells beginning to die.

Doses of sarin or soman which are multiples of the LD50 determined for the subject animals are administered subcutaneously (s.c.) in a volume of 0.5 ml/100 g body weight. The s.c. route is favored for parenteral administration to avoid first pass metabolism. Within one minute later, animals are administered 25 mg/kg 2-PAM and 20 mg/kg atropine sulfate intraperitoneally (i.p.). I.p. administration allows rapid administration of the agents and avoids damage to the leg muscle. Five minutes later either vehicle or one of four doses of a test compound is administered s.c. in a volume of 0.5 ml/100 g. Following such treatment, subject animals can be evaluated using one or more of the following tests to determine the effects produced by the present compounds.

Functional observational battery (FOB). Nerve agent symptoms to be evaluated include autonomic, neuromuscular and convulsive. Autonomic symptoms include eye closure and breathing status. Neuromuscular symptoms are primarily postural and gait. These include flattened posture, lying on side, prostrated and staggering. Convulsive symptoms include tail waving, tremors, and clonic convulsions or seizures. The FOB scores are taken every 15 minutes after nerve agent dosing. The minimum score for each animal is generally 5 (normal animal) and the maximum score is 21 (severely affected animal).

Locomotor activity. Locomotor activity can be evaluated in an automated open field system with infrared photo-beams (Motor Monitor, Version 3.11, 2000, Hamilton Kinder, Poway, Calif.). The open field is 16×16 inch (40.6×40.6 cm) and is divided into central and peripheral zones. The mice are placed in the center of the open field arena and the following variables of motor activity are recorded: locomotor activity, fine movement and rearing. In addition, distance traveled, total time, rest time, number of entries and head pokes in individual zones are recorded. All animals are regularly handled before individual tests in order to minimize handling-related stress. The animals are assigned to groups according to their basal locomotor activity, which is evaluated before any injections. After the session, the number of fecal pellets (defecation) is noted for assessment of emotional reactivity and the open field arena is cleaned.

Y maze activity. An acrylic maze test apparatus with 3 arms at 120 degrees to each other, each arm being 3.5 cm wide and 20 cm long, can be used to evaluate the effects of organophosphate exposure. Mice are acclimated to the room for 1 hr and then placed in one of the 3 arms. For the next eight minutes, they are video recorded for the sequence of arm entries, with an entry defined as all four paws within the arm. An alternation sequence is defined as entering three different arms in succession (e.g. ABC or BCA). The percentage of alternation is determined by dividing the total number of alternations by the total number of choices minus 2, multiplied by 100.

Body weight. Body weight loss after exposure to a nerve agent correlates with the extent of neuronal damage of a subject animal. A reduction in weight loss can therefore indicate a neuroprotective effect of one of the present compounds.

Stereological/Morphometric Analysis of Neuronal Cell Death. Brains of some subject animals are be removed and immersed in chilled isopentane to prepare them for further analysis. An initial coronal dissection can be made at 1.05 interaural, −2.75 Bregma. Coronal sections (10 μm) can be taken through 2.3 interaural, −1.2 bregma using a Leica crytotome. The serial sections can be collected on slides and stored until staining. Serial sections can be stained for one of the following: (1) cell death, using the TUNEL stain for apoptosis (Trevigen Inc., Gaithersburg, Md.); (2) GFAP (for astrocytes); or (3) mean cell density-nissl stain. Mean cell density can be determined by counting the stained nuclei using the Image-J image processing program.

To determine cell death in the brain, the TACS 2 TdT-Fluor In Situ Apoptosis Detection Kit TUNEL assay from Trevigen, Inc. can be used. Cryosectioned brain tissues are permeablized by incubating each section in Proteinase K Solution for 15 minutes followed by a 30 minute incubation in Cytonin. Sections are then washed and immersed in 1×TdT labeling buffer for 5 minutes and incubated for 1 hour at 37° C. with Labeling Reaction Mix. The labeling process is stopped by immersion in 1×TdT Stop Buffer for 5 minutes. Samples are then incubated in 0.5% Strep-Fluor Solution (or Strep-Cy2/5) 20. A positive control for apoptosis is created by incubating a section with TACS nuclease solution for 60 minutes immediately after treatment with Cytonin and Proteinase K. Images can be analyzed using current Image-J software.

E. Clinical Development

Following the testing of candidate compounds and/or compositions in preclinical animal models, candidates for further development can be selected based on the criteria set forth above. One or more selected candidates having desirable preclinical profiles can then be subjected to clinical evaluation in human patients using methods known to those of skill in the art.

### Treatments

The effects of nerve agent exposure can be prevented or ameliorated by administering therapeutically effective amounts of one or more of the present compounds and/or pharmaceutical compositions to a patient in need thereof. The present compounds and/or compositions are administered to a patient in a quantity sufficient to treat or prevent the symptoms and/or the underlying etiology associated with nerve agent exposure in the patient. The present compounds can also be administered in combination with other agents known to be useful in the treatment of nerve agent exposure, such as atropine sulfate, diazepam, and pralidoxime (2-PAM), either in physical combination or in combined therapy through the administration of the present compounds and agents in succession (in any order).

Administration of the present compounds and compositions can begin immediately following exposure to an organophosphate nerve agent, preferably within the first hour following exposure, and more preferably within one to five minutes. Administration of the compositions and compounds can alternatively begin prior to an anticipated exposure (such as impending combat), in order to prevent or reduce the impact of subsequent exposure. The present invention thus includes the use of the present compounds and/or a pharmaceutical composition comprising such compounds to prevent and/or treat exposure to a nerve agent.

Depending upon the particular needs of the individual subject involved, the present compounds can be administered in various doses to provide effective treatments for nerve agent exposure. Factors such as the activity of the selected compound, half life of the compound, the physiological characteristics of the subject, the extent or nature of the subject's exposure or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired. In view of the long-term effects of low-dose exposure to nerve agents, it is contemplated that repeated doses of the present compounds administered over an extended period of time may be required.

An effective amount of any embodiment of the present invention is determined using methods known to pharmacologists and clinicians having ordinary skill in the art. For example, the animal models described herein can be used to determine applicable dosages for a patient. As known to those of skill in the art, a very low dose of a compound, i.e. one found to be minimally toxic in animals (e.g., 1/10×LD10 in mice), can first be administered to a patient, and if that dose is found to be safe, the patient can be treated at a higher dose. A therapeutically effective amount of one of the present compounds for treating nerve agent exposure can then be determined by administering increasing amounts of such compound to a patient suffering from such exposure until such time as the patient's symptoms are observed or are reported by the patient to be diminished or eliminated.

In a preferred embodiment, the present compounds and compositions selected for use in treating or preventing nerve agent exposure have a therapeutic index of approximately 2 or greater. The therapeutic index is determined by dividing the dose at which adverse side effects occur by the dose at which efficacy for the condition is determined. A therapeutic index is preferably determined through the testing of a number of subjects. Another measure of therapeutic index is the lethal dose of a drug for 50% of a population (LD₅₀, in a pre-clinical model) divided by the minimum effective dose for 50% of the population (ED₅₀).

Blood levels of the present compounds can be determined using routine biological and chemical assays and these blood levels can be matched to the route of administration and half life of a selected compound. The blood level and route of administration can then be used to establish a therapeutically effective amount of a pharmaceutical composition comprising one of the present compounds for preventing and/or treating nerve agent exposure.

Exemplary dosages in accordance with the teachings of the present invention for these compounds range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the compound is being administered, the response to treatment, the type and quantity of other medications being given to the patient that might interact with the compound, either potentiating it or inhibiting it, and other pharmacokinetic considerations such as liver and kidney function.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

In addition, all groups described herein can be optionally substituted unless such substitution is excluded. Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group.

What is claimed is:

1. A method of treating the effects of exposure to an organophosphate compound, comprising administering to a subject in need thereof a therapeutically effective amount of a pharmaceutical composition comprising a compound having the following formula (Formula I): 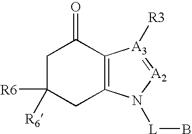
where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl; and
(d) R_{6′} is hydrogen unless R₆ is alkyl, in which case R_{6′} is hydrogen or the same alkyl as R₆.
(e) L is a linker; and
(f) B is a moiety having a formula selected from the group consisting of:
(i) Formula II: 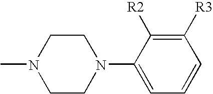
where:
(1) R2 is hydrogen, alkyl, hydroxy, halo, alkoxy, cyano, methylthio;
(2) R3 is hydrogen, alkyl, hydroxy, halo, alkoxy, trifluoromethyl, nitro, amino, aminocarbonyl, aminosulfonyl; and
(3) R2 and R3 can be taken together to form a 5 or 6 member aromatic or non-aromatic ring, which can contain from 0 to 3 heteroatoms selected from the group of N, O, or S of which the N may be further substituted if in a non-aromatic ring;
(ii) Formula III: 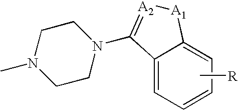
where:
(1) A¹ is N, O, or S, and when it is N, it can be further substituted with Z, which in alkyl, aralkyl, heteroaralky, or heteroalkyl.
(2) A2 is C or N; and
(3) R is selected from the group consisting of hydrogen, alkyl, NH₂, NHQ₁, NQ₁Q₂, OH, OQ₁, SQ₁, halo, nitro, cyano, and trifluoromethyl, and wherein Q₁ and Q₂ are selected from the group consisting of alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl; and
(iii) Formula IV: 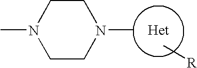
where the 6-member heterocyclic ring of Formula IV is selected from the group consisting of a 2-pyridyl moiety, a 4-pyridyl moiety, a 2-pyrimidyl moiety,
a 4-pyrimidyl moiety, a 2-pyrazinyl moiety, and a 2-triazinyl moiety; or a salt or ester thereof.

2. The method of claim 1, wherein the linker is selected from the group consisting of:
(a) a straight chain alkyl group having the formula —(CH₂)ₘ—, wherein m is an integer from 1 to 6; and
(b) an alkyl substituted hydrocarbyl moiety having the following formula: 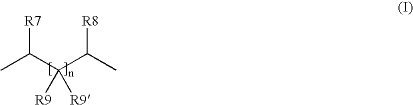
where:
(i)ₙ is 0, 1 or 2;
(ii) R7 and R8 are hydrogen, methyl or ethyl;
(iii) R9 and R9′ are both hydrogen, methyl or ethyl;
(iv) if n is 1 and R7 or R8 is methyl or ethyl, then R9 and R9′ are hydrogen;
(v) if n is 1 and R7 and R8 are hydrogen, then R9 and R9′ are methyl or ethyl; and
(vi) if n is 2, then R9 and R9′ are hydrogen and one or both of R7 and R8 are methyl or ethyl.

3. The method of claim 1, wherein:
(a) A₂ and A₃ are C;
(b) R₆ is hydrogen, alkyl, aralkyl, heteroaralkyl, aryl or heteroaryl; and
(c) R₆ and R₃ are hydrogen.

4. The method of claim 3, wherein R₆ is hydrogen.

5. The method of claim 1, wherein B is: 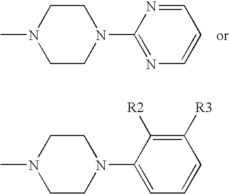
and R₂ and R₃ are the same or independently hydrogen, alkyl, hydroxy, halo, alkoxy, trifluoromethyl, nitro, amino, aminocarbonyl, or aminosulfonyl.

6. The method of claim 1, wherein B is a moiety selected from the group consisting of a m-trifluoromethylphenylpiperazinyl moiety, a m-chlorophenylpiperazinyl moiety, a o-methoxyphenylpiperazinyl moiety, a 1-naphthylpiperazinyl moiety, a 2-pyrimidylpiperazinyl moiety, a 3-indazolylpiperazinyl moiety a 2,3-dichlorophenylpiperazinyl moiety, and a 2,3-dimethylphenylpiperazinyl moiety.

7. The method of claim 1, wherein R is selected from the group consisting of a halo group, an alkyl group, a cyano group, a trifluoromethyl group, an alkoxy group, an amino group, an alkylamino group, and a dialkyamino group.

8. The method of claim 1, wherein the compound of Formula I is selected from the group consisting of:
1-{2-[4-(3-Trifluoromethylphenyl)piperazine-1-yl]ethyl}-1,5,6,7-tetrahydroindol-4-one;
1-{3-[4-(3-Trifluoromethylphenyl)piperazin-1-yl]propyl}-1,5,6,7-tetrahydroindol-4-one;
1-{4-[4-(3-Trifluoromethylphenyl)piperazin-1-yl]butyl}-1,5,6,7-tetrahydroindol-4-one;
1-{2-[4-(3-Chlorophenyl)piperazin-1-yl]ethyl}-1,5,6,7-tetrahydroindol-4-one;
1-{4-[4-(3-Chlorophenyl)piperazin-1-yl]butyl}-1,5,6,7-tetrahydroindol-4-one;
1-{2-[4-(2-Methoxyphenyl)piperazin-1-yl]ethyl}-1,5,6,7-tetrahydroindol-4-one;
1-{3-[4-(2-Methoxyphenyl)piperazine-1-yl]propyl}-1,5,6,7-tetrahydroindol-4-one;
1-{4-[4-(2-Methoxyphenyl)piperazin-1-yl]butyl}-1,5,6,7-tetrahydroindol-4-one;
1-{2-[4-(2-Pyrimidyl)piperazine-1-yl]ethyl}-1,5,6,7-tetrahydroindol-4-one;
1-{3-[4-(2-Pyrimidyl)piperazin-1-yl]propyl}-1,5,6,7-tetrahydroindol-4-one;
1-{4-[4-(2-Pyrimidyl)piperazin-1-yl]butyl}-1,5,6,7-tetrahydroindol-4-one;
1-{2-[4-(1-Naphthyl)piperazin-1-yl]ethyl}-1,5,6,7-tetrahydroindol-4-one;
1-{3-[4-(1-Naphthyl)piperazin-1-yl]propyl}-1,5,6,7-tetrahydroindol-4-one;
1-{4-[4-(1-Naphthyl)piperazin-1-yl]butyl}-1,5,6,7-tetrahydroindol-4-one;
1-{2-[4-(3-Indazolyl)piperazin-1-yl]ethyl}-1,5,6,7-tetrahydroindol-4-one;
1-{3-[4-(3-Indazolyl)piperazin-1-yl]propyl}-1,5,6,7-tetrahydroindol-4-one;
1-{4-[4-(3-Indazolyl)piperazin-1-yl]butyl}-1,5,6,7-tetrahydroindol-4-one;
1-{4-[4-(2,3-Dichlorophenyl)piperazin-1-yl]butyl}-1,5,6,7-tetrahydroindol-4-one;
1-{3-[4-(2,3-Dichlorophenyl)piperazin-1-yl]propyl}-1,5,6,7-tetrahydroindol-4-one;
1-{2-[4-(2,3-Dichlorophenyl)piperazin-1-yl]ethyl}-1,5,6,7-tetrahydroindol-4-one;
1-{4-[4-(2,3-Dimethylphenyl)piperazin-1-yl]butyl}-1,5,6,7-tetrahydroindol-4-one;
1-{3-[4-(2,3-Dimethylphenyl)piperazin-1-yl]propyl}-1,5,6,7-tetrahydroindol-4-one; and
1-{2-[4-(2,3-Dimethylphenyl)piperazin-1-yl]ethyl}-1,5,6,7-tetrahydroindol-4-one.

9. The method of claim 1, wherein the composition comprises a pharmaceutically acceptable excipient in combination with the compound of Formula I.

10. The method of claim 1, wherein the composition is administered by an administrative route selected from the group consisting of intravenous, oral, topical, intraperitoneal, intravesical, transdermal, nasal, rectal, vaginal, intramuscular, intradermal, subcutaneous and intrathecal.

11. The method of claim 1, wherein the therapeutically effective amount of the compound of Formula I is in the range of 0.0001 mg/kg to 60 mg/kg.

12. The method of claim 1, wherein the therapeutically effective amount of the compound of Formula I is administered to the subject following exposure of the subject to the organophosphate compound.

13. The method of claim 1, wherein the therapeutically effective amount of the compound of Formula I is administered to the subject prior to exposure of the subject to the organophosphate compound.
